(19) United States
(12) Patent Application Publication
(10) Pub. No.: US 2007/0167688 A1
(43) Pub. Date: Jul. 19, 2007

(54) Healthcare management systems and associated methods

(76) Inventors: S. Michael Ross, Merion Station, PA (US); Steve Zirkel, Concord, MA (US)

Correspondence Address: PERKINS COIE LLP;PATENT-SEA, P.O. BOX 1247, SEATTLE, WA, 98111-1247, US

(21) Appl. No.: 11/593,945

(22) Filed: Nov. 6, 2006

Related U.S. Application Data
(63) Continuation-in-part of application No. 11/311,917, filed on Dec. 19, 2005.

(51) Int. Cl. G06Q 10/00; A61B 5/00

(52) U.S. Cl. 600/300; 705/2

(57) ABSTRACT

The present invention relates to healthcare management systems and associated methods, including healthcare management methods in computing environments that provide prescription fulfillment management, care support services, patient opinion information, healthcare treatment adherence, and the ability to interface a healthcare management system with other systems. Certain aspects are directed toward a method for providing care support services related to one or more healthcare issues that includes collecting patient data, contacting at least one of the patients, and providing a care support service to the patient. Other aspects are directed toward a method for managing information regarding adherence to a treatment regimen that includes collecting patent data for each one of multiple patients, receiving an indication regarding at least one of the patient's adherence to a healthcare treatment regimen, and providing information regarding the indication to (a) the at least one patient, (b) a third party, or (c) both (a) and (b).

### CROSS-REFERENCE TO RELATED APPLICATIONS

This application is a continuation in part of U.S. patent application Ser. No. 11/311,917, entitled PRESCRIPTION CARE SUPPORT SERVICES, filed Dec. 19, 2005, which is hereby incorporated herein by reference in its entirety.

### TECHNICAL FIELD

The present invention relates to healthcare management systems and associated methods, including healthcare management methods in computing environments that provide prescription fulfillment management, care support services, patient opinion information collection, and the ability to interface a healthcare management system with other related systems.

### BACKGROUND

Managing the fulfillment of prescription medications and other prescription related services can be time-consuming and expensive for pharmaceutical providers. For example, pharmacist not only fill prescriptions, but must also be concerned with drug interaction issues, patient counseling, generic substitutions, dosage adherence issues, and drug recalls. Additionally, many pharmacists provide care support services related to various prescription medications, such as high-risk side effect surveillance and related healthcare education. Furthermore, in many instances the pharmacists acts as an interface between the patient, physician, and other entities regarding various prescription matters, including prescription renewals, insurance issues, and various patient concerns.

For many patients, customer service is an important factor in choosing a pharmacy and can also affect the overall quality of healthcare received by the patient. Accordingly, it is important for the pharmacist to understand which services are important to the pharmacist's patients and how the patients perceive the pharmacy is performing in various areas. Additionally, it is important that the pharmacist and the pharmacist's staff have the resources and time necessary to provide quality service. Therefore, it can be important to insure that the pharmacist and the pharmacist's staff are used effectively and efficiently in order to provide quality service and quality healthcare to the pharmacist's patients.

### BRIEF DESCRIPTION OF THE DRAWINGS

FIG. 1 is a block diagram that illustrates a computing environment suitable for implementing a healthcare management process in accordance with embodiments of the invention.

FIG. 2 is a flow diagram that illustrates a healthcare management process related to prescription fulfillment in accordance with various embodiments of the invention.

FIG. 3 is a flow diagram that illustrates a portion of a healthcare management process related to prescription fulfillment in accordance with selected embodiments of the invention.

FIG. 4 is a flow diagram that illustrates a portion of a healthcare management process related to prescription fulfillment in accordance with other embodiments of the invention.

FIG. 5 is a flow diagram that illustrates a portion of a healthcare management process related to prescription fulfillment in accordance with still other embodiments of the invention.

FIG. 6 is a flow diagram that illustrates a portion of a healthcare management process related to prescription fulfillment in accordance with yet other embodiments of the invention.

FIG. 7 is a flow diagram that illustrates a portion of a healthcare management process related to prescription fulfillment in accordance with still other embodiments of the invention.

FIG. 8 is a flow diagram that illustrates a portion of a healthcare management process related to prescription fulfillment in accordance with still other embodiments of the invention.

FIG. 9 is a flow diagram that illustrates a healthcare management process related to providing information requesting patient opinion data associated with a medication in accordance with embodiments of the invention.

FIG. 10 is a flow diagram that illustrates a portion of a healthcare management process that is related to providing information that reminds a patient to refill a prescription and requests patient opinion information associated with a medication in accordance with various embodiments of the invention.

FIG. 11 is a flow diagram that illustrates a portion of a healthcare management process that is related to providing information that reminds a patient to refill a prescription and requests patient opinion information associated with a medication in accordance with other embodiments of the invention.

FIG. 12 is a flow diagram that illustrates a healthcare management process for providing information regarding care support services related to prescription medication in accordance with embodiments of the invention.

FIG. 13 is a flow diagram that illustrates a portion of a healthcare management process for providing information regarding care support services related to prescription medication in accordance with various embodiments of the invention.

FIG. 14 is a flow diagram that illustrates a portion of a healthcare management process for providing information regarding care support services related to prescription medication in accordance with other embodiments of the invention.

FIG. 15 is a flow diagram that illustrates a portion of a healthcare management process for providing information regarding care support services related to prescription medication in accordance with still other embodiments of the invention.

FIG. 16 is a flow diagram that illustrates a process for providing formatted prescriptions related data for use in a healthcare management element in accordance with certain embodiments of the invention.

FIG. 17 is a partially schematic illustration of a healthcare management system in accordance with embodiments of the invention.

FIG. 18 is a flow diagram that illustrates a healthcare management process for providing care support services related to one or more healthcare issues including healthcare treatment regimens in accordance with embodiments of the invention.

FIG. 19 is a flow diagram that illustrates a healthcare management process related to collecting opinion data in accordance with embodiments of the invention.

### DETAILED DESCRIPTION

In the following description, numerous specific details are provided in order to give a thorough understanding of embodiments of the invention. One skilled in the relevant art will recognize, however, that the invention may be practiced without one or more of the specific details, or with other methods, components, materials, etc. In other instances, well known structures, materials, or operations are not shown or described in order to avoid obscuring aspects of the invention.

References throughout the specification to “one embodiment” or “an embodiment” means that a particular feature, structure, or characteristic described in connection with the embodiment is included in at least one embodiment of the present invention. Thus, the appearances of the phrase “in one embodiment” or “in an embodiment” in various places throughout the specification are not necessarily all referring to the same embodiment. Furthermore, the particular features, structures, or characteristics may be combined in any suitable manner in one or more embodiments.

The present invention is directed generally toward healthcare management systems and associated methods, including healthcare management methods in computing environments that provide prescription fulfillment management, care support services, patient opinion information collection, and the ability to interface a healthcare management system with other related systems. Aspects of the invention are directed toward a method for managing prescription medication fulfillment that includes collecting prescription related data for each one of multiple patients. Each patient can be associated with one or more prescriptions. The prescription related data can include a patient identification and/or one or more drug names associated with the one or more prescriptions. The method can further include contacting at least one of the patients and providing information about fulfillment of the patient's one or more prescriptions. In certain embodiments, the method can further include providing one or more response options regarding the fulfillment of the patient's one or more prescriptions and receiving an input indicating a selection of at least one of the one or more response options. In selected embodiments, the method for managing prescription medication fulfillment can include a method in a computing environment. In other embodiments, the method can include providing information to at least one of the patients via phone.

Other aspects of the invention are directed toward a method for providing care support services related to prescription medication, wherein the method includes collecting prescription related data for each one of multiple patients. Each patient can be associated with one or more prescriptions. The prescription related data can include a patient identification and/or one or more drug names associated with the one or more prescriptions. The method can further include contacting at least one of the patients and providing a care support service to the patient related to the patient's one or more associated prescriptions. In certain embodiments, the method can include providing one or more response options related to the care support service and receiving an input indicating a selection of at least one of the one or more response options. In selected embodiments, the method for providing care support services related to prescription medication can include a method in a computing environment. In other embodiments, the method can include providing a care support service to the patient via phone.

Still other aspects of the invention are directed toward a method for providing care support services related to one or more healthcare issues that includes collecting patient data for each one of multiple patients. The patient data includes a patient identification and/or information regarding one or more healthcare issues. The method further includes contacting at least one of the patients and providing one or more response options regarding the at least one patient's one or more healthcare issues. The method still further includes receiving an input indicating a selection of at least one of the one or more response options, and providing information regarding the selection of at least one of the one or more response options to (a) the at least one patient, (b) a third party, or (c) both (a) and (b).

Yet other aspects of the invention are directed toward a method for providing care support services related to one or more healthcare issues that includes collecting patient data for each one of multiple patients. The patient data includes at least one of a patient identification and information regarding one or more healthcare issues. The method further includes contacting at least one of the patients and providing a care support service to the patient related to the patient's one or more healthcare issues.

Still other aspects of the invention are directed toward a method for managing information regarding adherence to a treatment regimen that includes collecting patent data for each one of multiple patients. The patient data includes at least one of a patient identification and information regarding one or more healthcare issues. The method further includes receiving an indication regarding at least one of the patient's adherence to a non-medication treatment regimen and providing information regarding the indication to (a) the at least one patient, (b) a third party, or (c) both (a) and (b).

Yet other aspects of the invention are directed toward a method in a computing environment for managing information regarding adherence to a treatment regimen that includes collecting patient data for each one of multiple patients. The patient data includes at least one of a patient identification and information regarding one or more healthcare issues. The method further includes receiving an indication regarding at least one of the patient's adherence to a medication treatment regimen and providing information regarding the indication to (a) the at least one patient, (b) a third party, or (c) both (a) and (b).

Still other aspects of the invention are directed toward a method of collecting patient opinion information associated with a prescription medication, wherein the method includes collecting prescription related data for each one of multiple patients. Each patient can be associated with one or more prescriptions. The prescription related data can include a patient identification and/or one or more drug names associated with the one or more prescriptions. The method can further include contacting at least one of the patients and providing one or more response options to the patient regarding a request for patient opinion information. The method can still further include receiving an input from the patient indicating a selection of at least one of the one or more response options and providing an output based on the patients' selection of at least one of the one or more response options. In certain embodiments, the method of collecting patient opinion information includes a method in a computing environment.

Yet other aspects of the invention are directed toward a method for providing formatted prescription related data for use in a healthcare management element that includes collecting raw prescription related data for each one of multiple patients. Each patient can be associated with one or more prescriptions. The prescription related data can include a patient identification and/or one or more drug names associated with the one or more prescriptions. The method can further include processing the raw prescription related data for each one of the multiple patients to produce formatted prescription related data. The method can still further include providing the formatted prescription related data for each one of the multiple patients to a healthcare management element. In selected embodiments, the method can further include collecting drug related information. In certain embodiments, the method for providing formatted prescription related information includes a method in a computing environment.

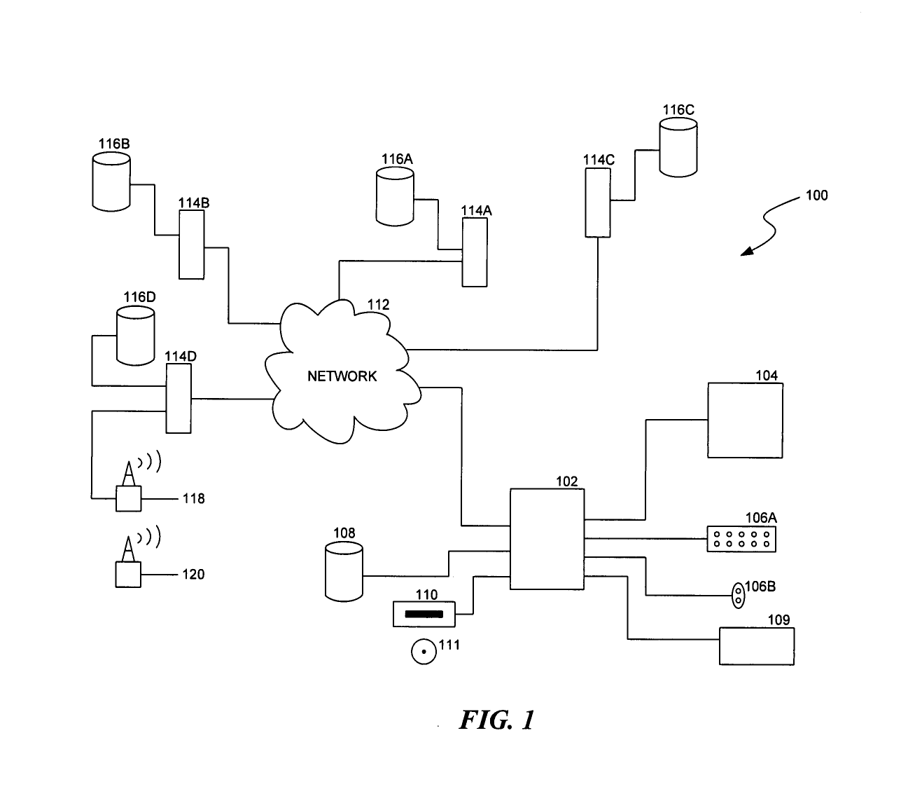

FIG. 1 is a block diagram that illustrates a computing environment suitable for implementing a process related to a healthcare management system in accordance with embodiments of the invention. The computing environment 100 can include a computing or computer system 102 that can be operably connected or coupled to a display 104 and one or more input devices, for example, a keyboard 106a and a pointing device 106b (e.g., a mouse). Additionally, the computer system 102 can communicate with one or more storage devices (e.g., a hard drive 108 with one or more databases) and one or more devices 110 for reading other types of computer readable mediums (e.g., devices for reading disks 111). The computing system 102 can also communicate directly with other devices 109, for example, a phone and/or fax system, or with other devices or systems via a network 112 (e.g., via the Internet). For example, in the illustrated embodiment the computer system 102 can communicate with other computer systems 114a-d and/or other databases 116a-d via the network 112. Additionally, in selected embodiments the computing system can communicate with wireless devices 120 (e.g., a wireless computing device such as a personal data assistant) via a wireless transmitter/receiver (e.g., a service provider connected to the internet). In other embodiments, the computing environment can have other arrangements, including more, fewer, and/or different components.

For example, the computing device or environment on which the system is implemented may include a central processing unit, memory, input devices (e.g., keyboard and pointing devices), output devices (e.g., display devices), and storage devices (e.g., disk drives). The memory and storage devices are computer-readable media that may contain instructions that implement the system. In addition, the data structures and message structures may be stored or transmitted via a data transmission medium, such as a signal on a communication link. Various communication links may be used, such as the Internet, a local area network, a wide area network, a point-to-point dial-up connection, a cell phone network, and so on.

Embodiments of the system may be implemented in various operating environments that include personal computers, server computers, hand-held or laptop devices, multiprocessor systems, microprocessor-based systems, programmable consumer electronics, digital cameras, network PCs, minicomputers, mainframe computers, distributed computing environments that include any of the above systems or devices, and so on. The computer systems may be cell phones, personal digital assistants, smart phones, personal computers, programmable consumer electronics, digital cameras, and so on.

The system may be described in the general context of computer-executable instructions, such as program modules, executed by one or more computers or other devices. Generally, program modules include routines, programs, objects, components, data structures, and so on that perform particular tasks or implement particular abstract data types. Typically, the functionality of the program modules may be combined or distributed as desired in various embodiments.

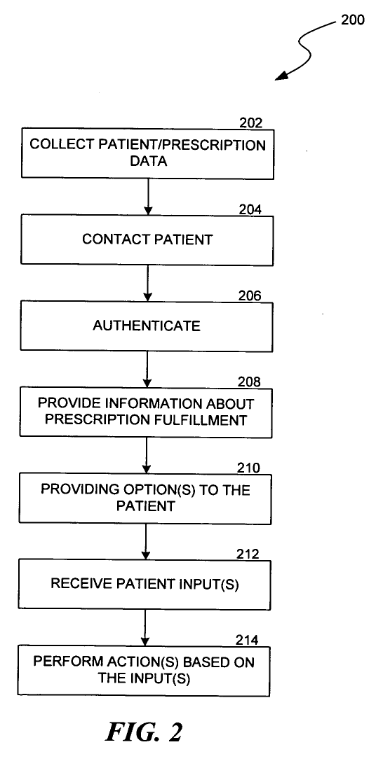

FIG. 2 is a flow diagram that illustrates a healthcare management process related to prescription fulfillment 200 in accordance with various embodiments of the invention. In FIG. 2, the prescription medication fulfillment process 200 includes collecting prescription related data for one or more patients (process portion 202). For example, in certain embodiments each patient can be associated with one or more prescriptions and in the prescription related data can include the patient's identification and/or one or more drug names associated with the one or more prescriptions.

In selected embodiments, the prescription related data can be collected by a medical services facility, such as a pharmacy, insurance company, health maintenance organization, doctor's office, or other health care related organization when a patient submits a prescription for fulfillment. In certain embodiments, prescription related data collected by the medical services facility can be subsequently entered into and collected by the healthcare management system. In other embodiments, prescription related information can also include other types of information and/or be obtained via other methods. For example, various types of prescription related data can include information about various medications obtained from a drug information database and/or historical patient information obtained from an insurance database.

The fulfillment process 200 can further include contacting at least one patient (process portion 204), authenticating the identity of the at least one patient (process portion 206), and providing information to the at least one patient about fulfillment of the patient's one or more prescriptions (process portion 208). For example, in certain embodiments providing information to the patient(s) can include providing information about a time to refill one or more prescriptions, a delay in filling one or more prescriptions, a recall of one or more prescriptions, a generic substitution of one or more prescriptions, a generic substitution option regarding one or more prescriptions, the status of the fulfillment of one or more prescriptions, and/or a notice regarding a last refill on one or more prescriptions. In other embodiments, providing information can include an offer to enroll patient(s) in an automatic refill program that automatically refills the patient(s) one or more prescriptions when the patient(s) is expected to run out of medication. In other embodiments, providing information can include providing notice that a filled prescription is ready for pickup, or that the filled prescription will be ready for pickup during a selected period of time. Additionally, in certain embodiments providing information can include providing a patient with the option to have a filled prescription delivered.

In other embodiments, the fulfillment process 200 can include providing the patient(s) with one or more response options regarding the fulfillment of one or more prescriptions (process portion 210) and receiving an input indicating a selection of at least one of the one or more response options (process portion 212). In still other embodiments, the fulfillment process 200 can include performing an action based on the input received (process portion 214). For example, in certain embodiments the input indicating a selection of one or more response options can be reported or sent to a pharmacy, pharmacist, pharmacy staff member, other medical service provider (e.g., health maintenance organization, insurance company, hospital, a doctor, doctor's office, a doctor's staff, and/or other healthcare related organization). In still other embodiments, the input indicating a selection of one or more response options can be stored for future use (e.g., to send a reminder to a pharmacist or pharmacy staff member that it is time to refill a prescription based on an automatic refill selection). In various embodiments, some, all, or none of the process portions discussed above can be performed in a computing environment.

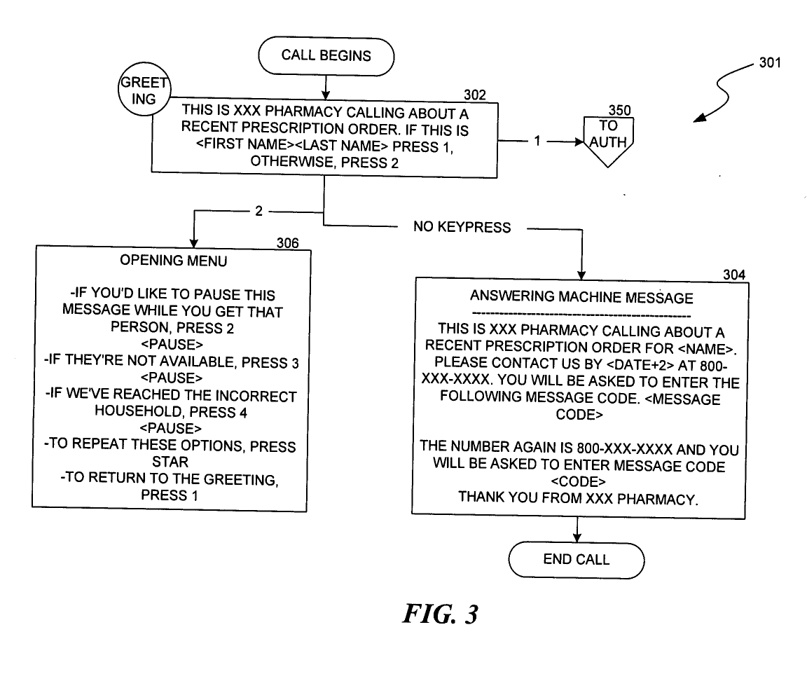

FIG. 3 is a flow diagram that illustrates a portion of a healthcare management process related to prescription fulfillment in accordance with selected embodiments of the invention. In FIG. 3, prescription related data has been collected and a phone call is placed to a patient. For example, the phone call can be placed by a healthcare management system that includes a computing system similar to the computing system 100 discussed above with reference to FIG. 1. When the phone call is answered, the greeting process 301 begins by playing a greeting message (process portion 302) that provides the person answering the phone with an option to transfer to an authentication process 350 via a first phone key selection or to transfer to an opening menu process 306 via a second phone key selection. If there is no key selection, a message requesting that the patient contact the pharmacy and/or a healthcare management system (e.g., via phone) is played for recordation on an answering machine (process portion 304). From the opening menu process 306 the person answering the phone can select multiple response options, including returning to the greeting message.

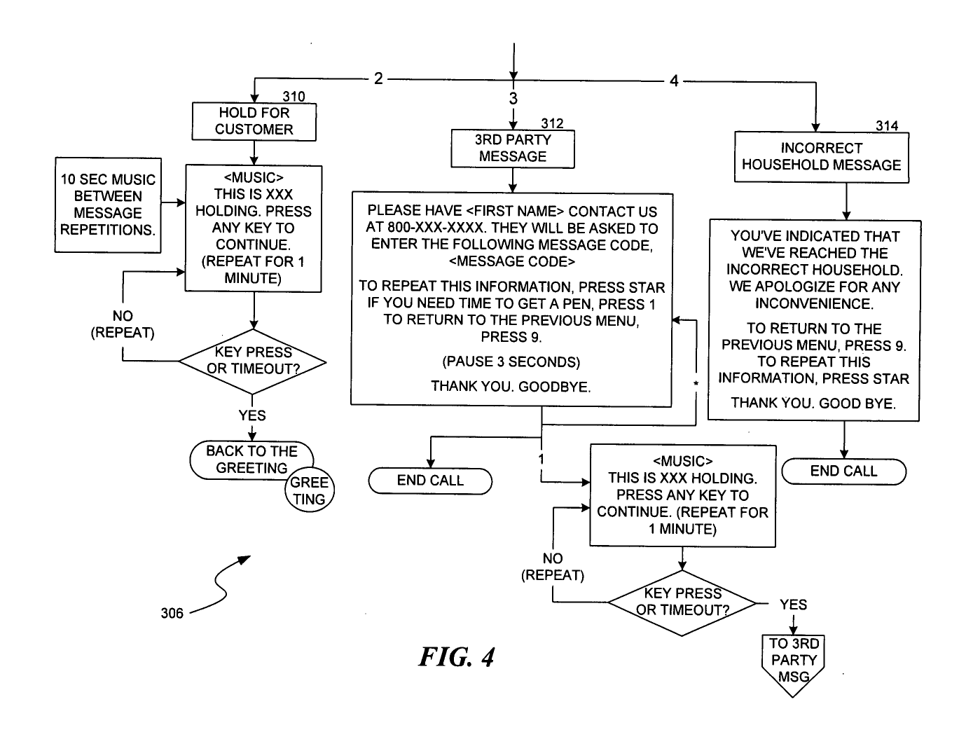

FIG. 4 is a flow diagram that illustrates additional portions of the opening menu process 306 corresponding to various phone key selections shown in FIG. 3. For example, if the person answering the phone selects “2” in the opening menu process 306, shown in FIG. 3, the hold process 310 is initiated allowing the person answering the phone to return to the greeting process after a selected period of time. If the person answering the phones selects “3” in the opening menu process 306, shown in FIG. 3, the third-party message process 312 is initiated and provides the person answering the phone with a message requesting that the person answering the phone to have the patient contact the pharmacy and/or a healthcare management system. In certain embodiments, the third-party message process 312 and/or the message played for the answering machine can provide an access code or other identification for the patient to use when contacting the pharmacy and/or the healthcare management system. If the person answering the phones selects “4” in the opening menu process, shown in FIG. 3, the incorrect household process 314 is initiated playing a message that apologizes for any inconvenience and also provides the person answering the phone the option to return to the previous menu.

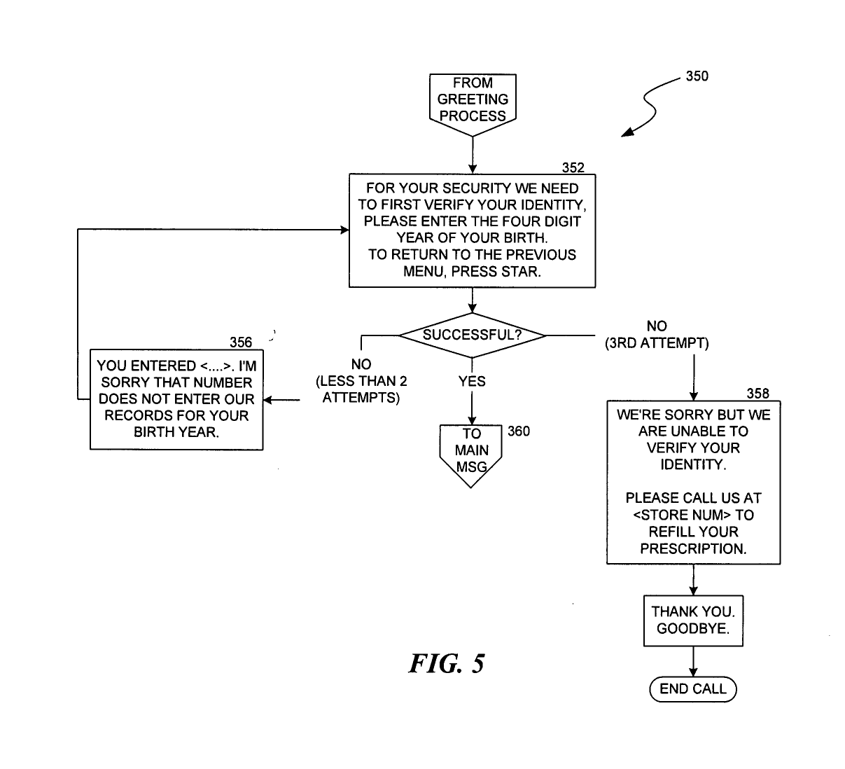

FIG. 5 is a flow diagram that illustrates the authentication process 350, discussed above with reference to FIG. 3. The authentication process 350 can be used to authenticate an identity of a patient. The authentication process can serve various functions, including providing patient privacy, providing security, and/or meeting regulatory requirements. In the illustrated embodiment, the authentication process 350 includes a request that the patient enter some form of identification (process portion 352). Various forms of identification can be used, including a birth date, a birth year, an access code, or other identifying information. Alphanumeric forms of identification can be particularly useful when inputting the identification via phone key selection. However, in other embodiments other forms of identification can be used (e.g., voice-recognition) and/or other authentication processes can be used. In still other embodiments, an authentication process is not used (e.g., when the patient is contacted via email or instant messaging).

In the illustrated embodiment, if the person answering the phone enters incorrect identification information, the person is provided with additional opportunities to enter the correct information (process portion 356). If no identification information is entered or a selected number of incorrect entries are made, the person answering the phone is provided a phone number to call for assistance (process portion 358). If the correct identification information is entered within the allowed number of attempts, the person answering the phone is transferred to the main message process 360, which can provide information and/or various options to the patient. As used herein, the term “patient” refers to a person for whom a corresponding prescription is written and/or that person's representative(s). For example, if the prescription related information includes a prescription written for a child, the patient can include the child and/or the child's parent(s) or guardian(s).

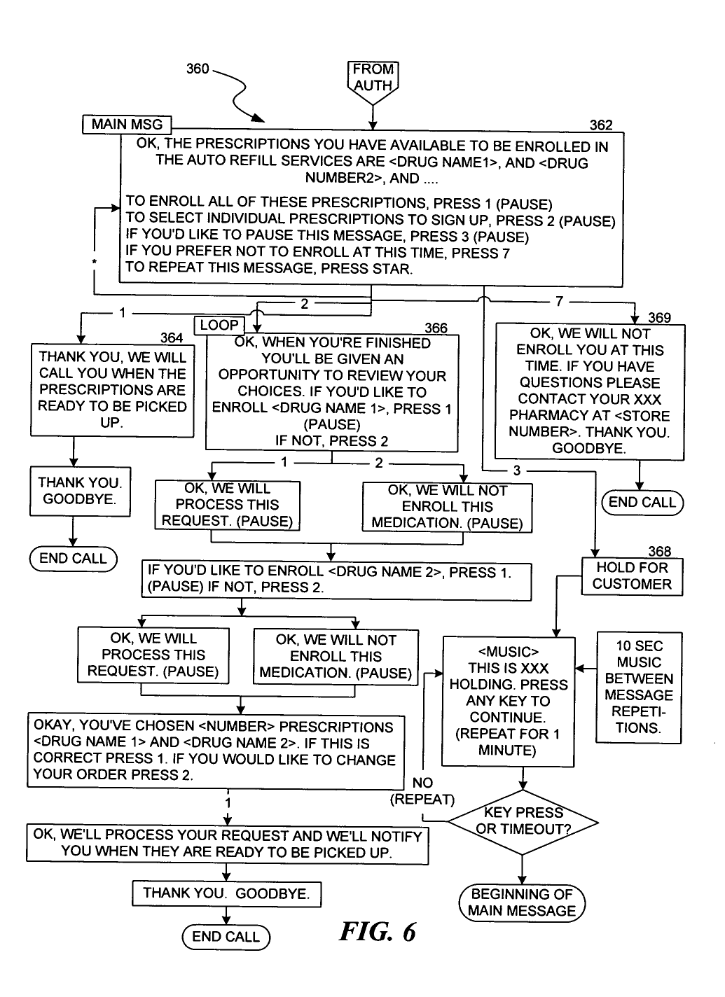

FIG. 6 is a flow diagram that illustrates an embodiment of the main message process 360 discussed above. In certain embodiments, the main message process can provide the patient with information regarding prescription fulfillment, information regarding care support information related to one or more prescriptions, and/or information regarding a request for additional information from the patient. In FIG. 6, the main message process 360 provides the patient with the opportunity to have one or more prescriptions automatically refilled at appropriate time(s) over the time period that the prescription is valid For example, the prescription related data can include one or more new prescriptions for a patient that have been received by a pharmacy and the healthcare management system can contact the patient to see if the patient would like to have one or more of the new prescriptions automatically refilled when the patient would be expected to have run out of the corresponding medication (e.g., once a month or once every 90 days depending on the quantity dispensed each time the prescription is filled).

In the illustrated embodiment, the main message process 360 includes a main message 362 that informs the patient that one or more prescriptions are eligible for enrollment in an automatic refill program and provide the patient with various response options that are selectable by pressing one or more phone keys. For example, in FIG. 6 the patient can indicate that the patient wishes to have all eligible prescriptions enrolled in the automatic refill service (process portion 364) or none of the eligible prescriptions enrolled in the automatic refill service (process portion 369). The patient can also indicate that the patient wishes to have only selected eligible prescriptions enrolled in the automatic refill service (process portion 366). In the illustrated embodiment, the selection of prescriptions to be enrolled in the automatic refill program can be made via additional phone key selections in response to various options presented by the healthcare management system. Additionally, in FIG. 6 the patient can pause the message (process portion 368), for example, in order to get pen, paper, and/or additional information necessary to make an automatic refill enrollment decision.

Once the patient selects one or more response options, the healthcare management system can take one or more additional actions. For example, the patient's response options selections can be stored, processed, and/or reported to an entity, such as a pharmacy or a pharmacy staff member (including a pharmacist). In certain embodiments, the automatic refill enrollment of a selected prescription can be processed and the healthcare management system can provide a reminder (e.g., to a pharmacy) when it is time to refill the selected prescription.

Although in FIG. 6 the main message provides a patient with the ability to enroll eligible prescription(s) in an automatic refill service, in other embodiments the main message can provide other kinds of information. For example, in selected embodiments the main message can inform the patient that the doctor has authorized a generic substitution for the prescribed medication and that the prescription will be filled with the generic drug. In other embodiments, the main message can provide the patient with a selectable response option to have a generic drug substituted, if authorized by the prescription, and/or to request that the pharmacy contact the prescribing physician to request a generic substitution.

In still other embodiments, the main message can “thank” the patient for placing a new prescription order with the pharmacy, offer the patient the option to enroll in the automatic refill service, and provide options regarding care support services and/or patient opinion information (both discussed below in further detail). For example, in certain embodiments the main message can offer the patient the option to enroll in the automatic refill service along with the option to be immediately connected with a pharmacist for a consultation regarding the prescription and/or the option to complete a survey related to the prescription. The survey can query the patient for information related to the prescription (e.g., concerns about drug side effects, concerns about prescription costs, and/or concerns about the relationship of the drug to a specific illness). Based on the patient's response to the survey, the pharmacist can call the patient to discuss specific issues. This feature can be especially useful for serving new therapy patients. Additionally, if the patient declines to have the prescription automatically refilled, the patient can be provided additional survey questions regarding the reasons for declining this option. In yet other embodiments, the healthcare management system can provide still other information.

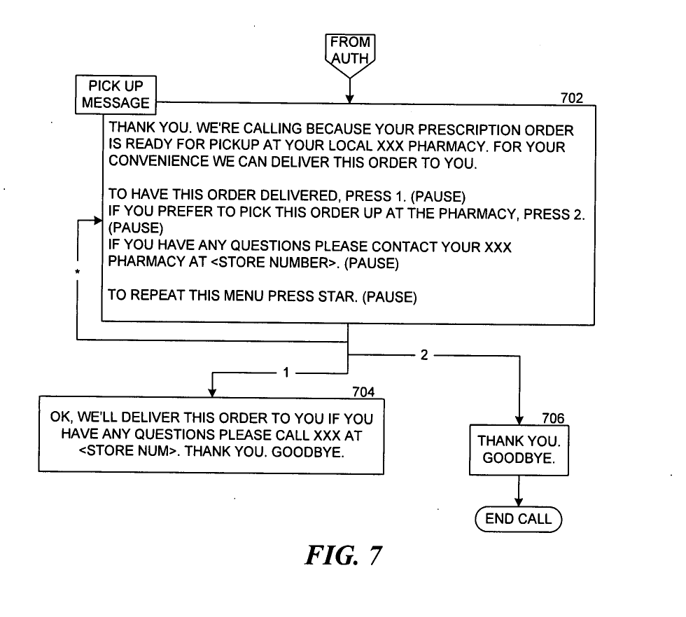

For example, FIG. 7 is a flow diagram that illustrates a portion of a healthcare management process related to prescription fulfillment in accordance with still other embodiments of the invention. In FIG. 7, after a patient has been authenticated, a pickup message (e.g., similar to the main message discussed above with reference to FIG. 6) is provided to the patient (process portion 702) to inform the patient that one or more prescriptions have been filled and are ready for pickup and/or delivered. In selected embodiments, the pickup message can provide the patient with the option to indicate that the one or more filled prescriptions will be picked up at the pharmacy or to have the one or more filled prescriptions delivered. In other embodiments, the pickup message simply informs the patient that the prescription is ready for pickup. In selected embodiments, the selected response option can be reported to the corresponding pharmacy so that the pharmacy can take appropriate action.

In the illustrated embodiment, if the patient indicates that the prescription will be picked up at the pharmacy the healthcare management system thanks the patient and ends the call (process portion 706). If the patient selects the delivery option, the healthcare management system provides confirmation that the prescription will be delivered (process portion 704). In certain embodiments, if the patient selects the delivery option, the healthcare management system can provide additional response options related to the payment method the patient expects to use when the medication is delivered.

The pickup message, shown in FIG. 7, can be used to indicate that a filled prescription is ready for pickup whether the prescription was filled automatically or not. In still other embodiments, the healthcare management system can provide additional information and/or options associated with a filled prescription. For example, in selected embodiments the healthcare management system can notify a patient that a filled prescription is ready for pickup and that the last refill of the prescription has been used. Additionally, in certain embodiments the healthcare management system can also provide the patient with an option to have the pharmacy contact the patient's physician to have a filled prescription renewed (e.g., when the last refill has been used to fill the current prescription).

In still other embodiments, a pickup message can inform a patient of the hours during which the pharmacy open and during which a filled prescription is available for pickup. Additionally, in certain embodiments the pickup message can provide a patient with a store location and/or a store phone number. In yet other embodiments, the pickup message can inform a patient that a prescription will be available for pickup only during a selected period of time. In still other embodiments, when a filled prescription has not been picked up during a first selected period of time, the healthcare management system can notify a patient that the filled prescription will only be available for pickup during a second selected period of time, and if the prescription is not picked up during the second selected period of time, the patient will have to place a new request with the pharmacy for the prescription. Although in the illustrated embodiment, the pickup message 702 is provided to the patient after patient authentication, in other embodiments the healthcare management system calls the patient and provides the associated pickup information without patient authentication. In still other embodiments, the healthcare management system can provide other types of information to the patient.

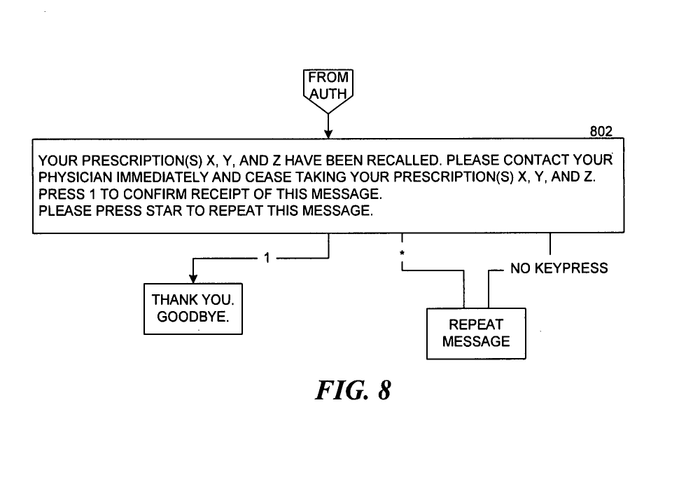

For example, FIG. 8 is a flow diagram that illustrates a portion of a healthcare management process related to prescription fulfillment in accordance with yet other embodiments of the invention. In FIG. 8, after patient authentication, an informational message 802, similar to the main message discussed above with reference to FIG. 6, is provided to inform a patient that one or more prescription medications associated with the patient have been recalled. In certain embodiments, the informational message 802 can provide additional information and/or one or more options. For example, in the illustrated embodiment the informational message 802 provides a response option for use by the patient to confirm receipt of the recall notification. Additionally, in the illustrated embodiment, the informational message includes instructions to cease taking the medication and to contact the patient's physician immediately. In other embodiments, other instructions can be provided. In selected embodiments, the healthcare management system can track the receipt of the recall notification and provide a summary of information to the corresponding pharmacy.

In still other embodiments, the informational message 802 to can include other types of information. For example, the informational message can include notification that it is time to fill a prescription, that fulfillment of a prescription has been delayed, a status of a prescription (e.g., information that the pharmacy has contacted the patient's physician about a generic substitution but the physician has not yet responded), a separate independent notice that there are no refills available on a selected prescription (e.g., because the authorized number of refills has been reached and/or a selected time period has expired), and/or information regarding the potential substitution of various generic drugs.

As discussed above, the healthcare management system can include a computing system and some, all, or none of the healthcare management process can be performed in a computing environment. Additionally, although embodiments of the healthcare management method discussed above with reference to FIGS. 3-8 have been described using a phone key selection interface to receive patient selections, those skilled in the art will understand that in other embodiments other methods of interfacing with the patient can be used. For example, in other embodiments the healthcare management system can use voice recognition, email, fax, websites, and/or instant messaging to interface with patients.

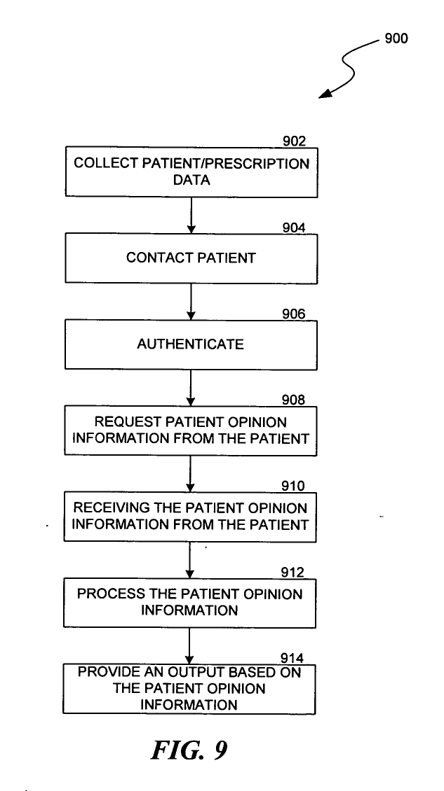

FIG. 9 is a flow diagram that illustrates a healthcare management process related to collecting patient opinion information from a patient associated with a medication 900 in accordance embodiments of the invention. The process in FIG. 9 can include collecting prescription related data for each one of multiple patients (process portion 902) and contacting at least one of the patients (process portion 904). Each patient can be associated with one or more prescriptions and prescription related data can include at least one of the patient's identification and one or more drug names associated with the one or more prescriptions. The process 900 can further include authenticating an identity of at least one patient (process portion 906). In certain embodiments, the process of collecting prescription related data, the process of contacting at least one patient, and the process of the authenticating an identity of the at least one patient can be similar to the corresponding processes discussed above with reference to FIGS. 3-8.

The process 900 can further include providing information that requests patient opinion information or data associated with a prescription medication from the at least one patient (process portion 908). For example, in certain embodiments requesting patient opinion information can include a request for feedback about a pharmacy, a service provided by a pharmacy, a medical service related to one or more prescriptions, and/or a patient's decision to have a prescription filled or not filled. In various embodiments, the request for patient opinion information can include one or more response options presented to the patient.

The process 900 can also include receiving the patient opinion information from the at least one of the patient (process portion 910). For example, receiving the patient opinion information can include receiving an input indicating a selection of one or more response options presented to the patient. The process 900 can further include processing the patient opinion information (process portion 912). For example, the patient opinion information received from one or more patients can be summarized and/or analyzed to provide a more meaningful format or output. In one embodiment, the reasons that patients do not have prescriptions refilled at a selected pharmacy can be tabulated to show the various reasons and the number of patients corresponding to each reason. In still other embodiments, the process 900 can include providing an output based on the patient opinion information (process portion 914). For example, the various patient inputs and/or the processed data based on patient input can be provided to a corresponding pharmacy that has filled a related prescription.

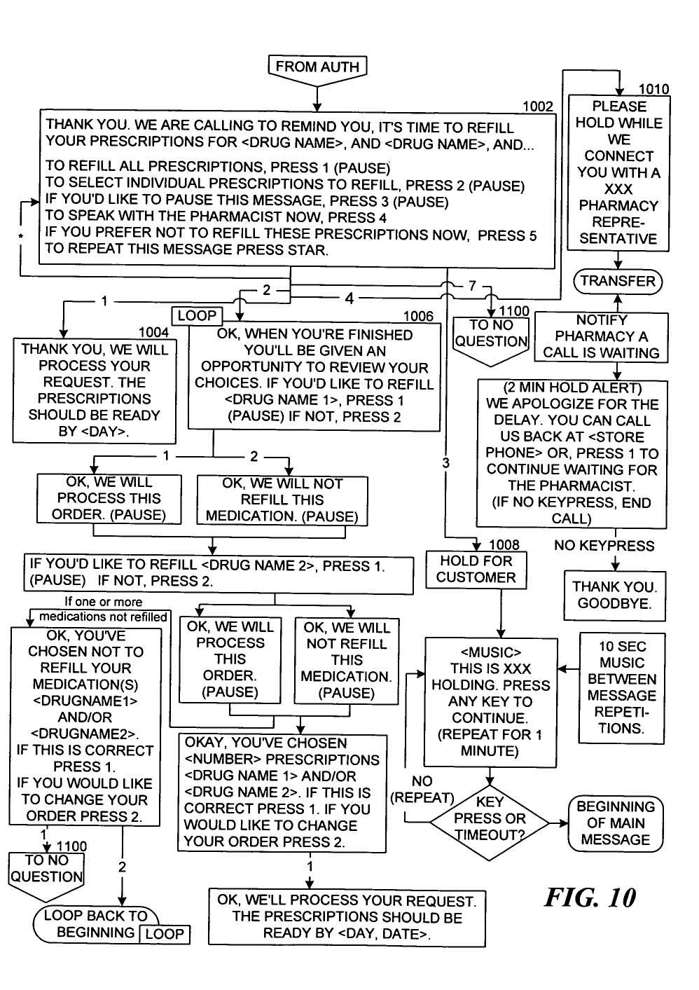

For the purpose of illustration, selected embodiments of a process for collecting patient opinion information will be discussed in combination with a process for reminding a patient to refill a prescription. It will, however, be understood by those skilled in the art that these two processes can be practiced individually and/or in combination with other processes is discussed herein. FIG. 10 is a flow diagram that illustrates a portion of a healthcare management process that is related to providing information that reminds a patient to refill a prescription and requests patient opinion information associated with a medication in accordance with various embodiments of the invention. In FIG. 10, after a patient has been contacted and authenticated (e.g., similar to the process is discussed above with reference to FIGS. 3-8), the healthcare management system can provide a main message reminding a patient that it is time to refill a prescription 1002. In certain embodiments, the reminder message 1002 can include one or more response options allowing the patient to have one or more prescriptions filled. For Example, in FIG. 10 the patient can select various response options via a key press on a phone. The patient can choose to have all prescriptions refilled (process portion 1004), selected prescriptions refilled (process portion 1006), or no prescriptions refilled. Additionally, in the illustrated embodiment the patient can pause the message (process portion 1008) and/or request to be connected to a pharmacy staff member (process portion 1010). In FIG. 10, if the patient chooses to not refill one or more prescriptions, a patient enters the no question process 1100.

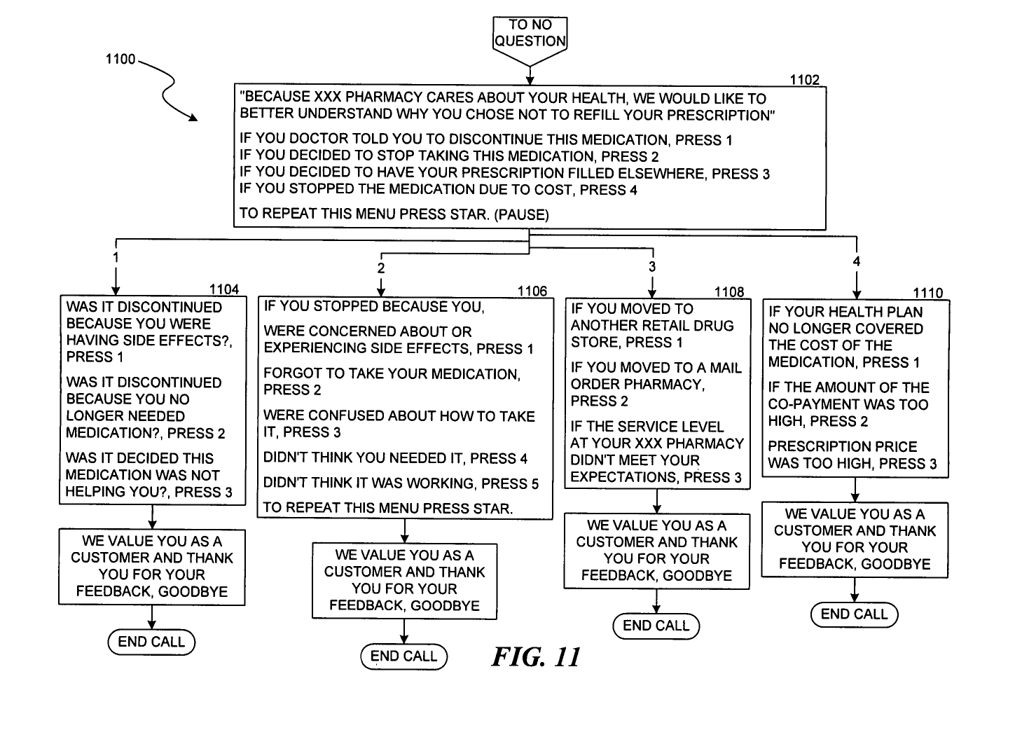

FIG. 11 is a flow diagram that illustrates the no question process 1100 in accordance various embodiments of the invention. In FIG. 11, the no question process 1100 includes a request for patient opinion information (process portion 1102) and provides a patient with one or more response options. If the patient selects the response option indicating that the doctor instructed the patient to discontinue the medication, the patient is presented with the doctor response process 1104. The doctor response process 1104 requests additional information from the patient and includes one or more response options to facilitate patient opinion information collection. If the patient indicates that it was the patient's decision to stop taking the medication, the patient is present with the patient response process 1106, which requests additional data. If the patient indicates that the patient has decided to have the prescription filled at a different pharmacy, the patient is presented with the pharmacy information process 1108, which requests opinion data regarding the original pharmacy. If the patient indicates that the patient has stopped taking the medication due to cost, the patient is presented with the cost information process 1110, which requests information regarding the cost of the medication. In other embodiments, the healthcare management system can collect other types of information regarding patient opinion.

As discussed above, the healthcare management system can include a computing system and some, all, or none of the information collection process discussed above with reference to FIGS. 9-11 can be performed in a computing environment. Additionally, although embodiments of the information collection process discussed above with reference to FIGS. 10-11 have been described using a phone key selection interface to receive patient input, those skilled in the art will understand that in other embodiments other methods of interfacing with the patient can be used. For example, in other embodiments the healthcare management system can use voice recognition, email, fax, websites, and/or instant messaging to interface with patients.

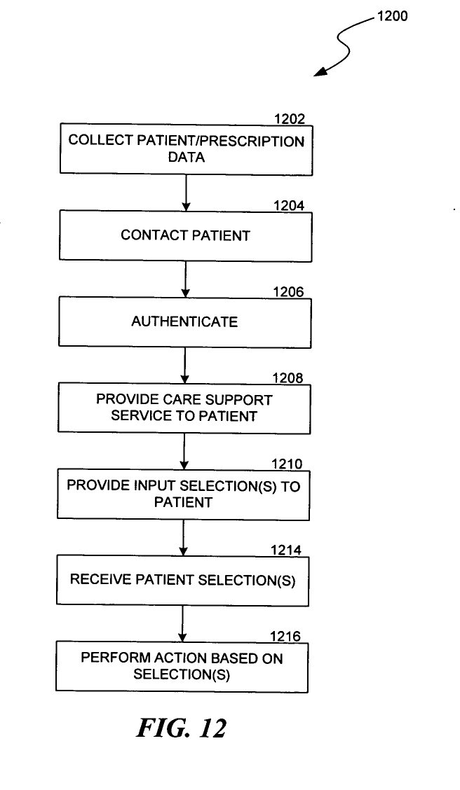

FIG. 12 is a flow diagram that illustrates a healthcare management process 1200 for providing care support services related to one or more healthcare issues in accordance with embodiments of the invention. For example, a healthcare issue can include a health condition (e.g., disease, injury, preventative treatments, and/or the like) that requires a treatment regimen, including prescription medication, non-prescription medication, physical therapy, exercise, thermal therapy (e.g., hot or cold pack), other types of medical treatment, and/or the like. Accordingly, the process 1200 in FIG. 12 can include collecting prescription related data for each one of multiple patients (process portion 1202) and contacting at least one of the patients (process portion 1204). Each patient can be associated with one or more prescriptions and prescription related data can include at least one of the patient's identification and one or more drug names associated with the one or more prescriptions. The process 1200 can further include authenticating an identity of at least one patient (process portion 1206). In certain embodiments, the process of collecting prescription related data, the process of contacting at least one patient, and the process of the authenticating an identity of the at least one patient can be similar to the corresponding processes discussed above with reference to FIGS. 3-8.

In the illustrated embodiment, the process 1200 further includes providing care support information to the at least one patient. In certain embodiments, the care support information can be related to the patient's one or more associated prescriptions. For example, the care support service information provided to the at least one patient can include, among other things, providing new medication counseling, a reminder to make a follow-up medical appointment related to a medication, a reminder to take a medication, a tip on how to remember taking a medication, health education related to a prescribed medication, testimonial information about a personal experience related to a medication or related medical condition, information related to drug dosage adherence monitoring, patient monitoring related to a medication, automatic consultation scheduling (e.g., scheduling an appointments with a medical service provider), phone reminders regarding scheduled consultation appointments, and/or information related to drug side effects. In certain embodiments, the care support information can include various response options that allow patient interaction so that the care support service information can be better tailored to the patient's needs.

In selected embodiments, the process 1200 can further include a request for input and/or one or more response options (process portion 1210). In other embodiments, the process 1200 can still further include receiving one or more inputs from the patient indicating a selection of one or more of the response options (process portion 1214). It still other embodiments, the process 1200 can further include performing an action based on one or more inputs received from the patient (process portion 1216). For example, in certain embodiments one or more of the inputs received from the at least one patient can be provided to a third party including a medical staff member (e.g., a pharmacy staff member and/or staff member at another type of medical facility) or other individual/organization related to the well being of the patient (e.g., a patient's family member). In other embodiments, the inputs received from the at least one patient can be summarized and provided to the at least one patient in the form of feedback (e.g., a “report card”).

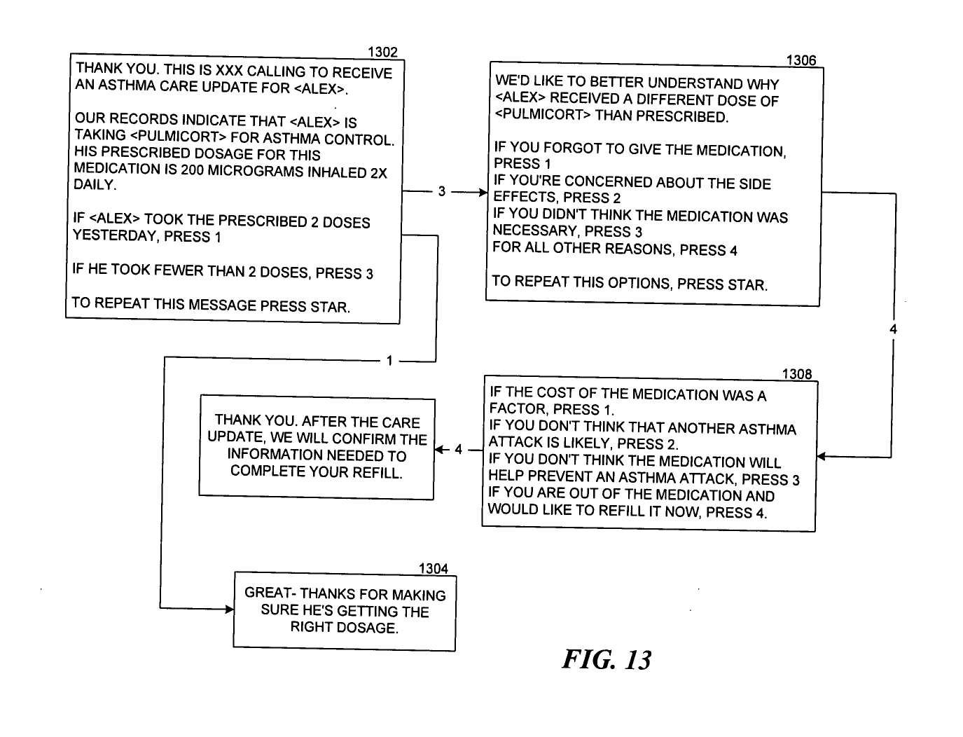

FIGS. 13-15 illustrate various embodiments of the Process 1200, shown in FIG. 12. Although in the illustrated embodiment these processes are shown linked together, in other embodiments the processes discussed in FIGS. 13-15 can stand alone or be combined with other processes described herein. FIG. 13 is a flow diagram that illustrates a portion of a healthcare management process for providing care support services related to prescription medication in accordance with various embodiments of the invention. In FIG. 13, the patient (e.g., the mother of the person for whom the prescription is written) is provided information regarding the prescription and requested to respond to multiple response options (process portion 1302). If the patient indicates (e.g., via telephone key presses) that the prescribed dosage has been taken, the healthcare management system provides acknowledgment (process portion 1304). If the patient indicates that less than the prescribed dosage was taken, the process management system queries the patient as to why a lesser dosage was taken in process portions 1306 and 1308. In certain embodiments, the patient input received can be provided to a pharmacy or other medical service provider. Accordingly, medication dosage adherence can be monitored and assistance can be provided where necessary. Additionally, this process can serve as a reminder to take the medication as prescribed.

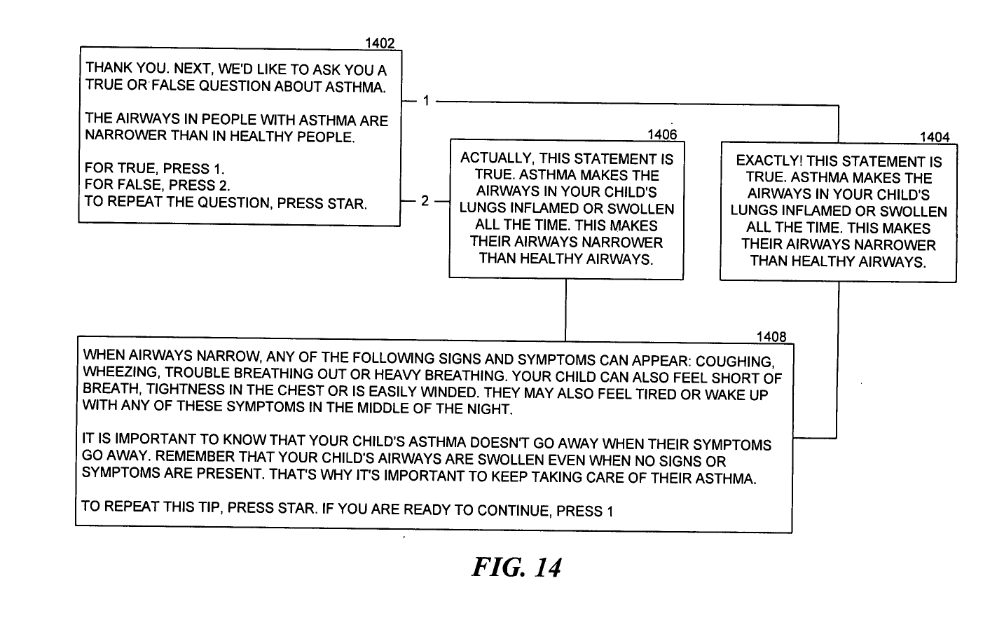

In FIG. 14 the healthcare management system queries a patient about general medical information related to the prescription that has been prescribed (process 1402). The patient is then provided feedback depending on the response option the patient selects (process portions 1404 and 1406). In either case, the patient is provided with additional information regarding additional general medical information related to the prescription (process portion 1408). Accordingly, the patient receives health education about a general medical condition related to a prescription.

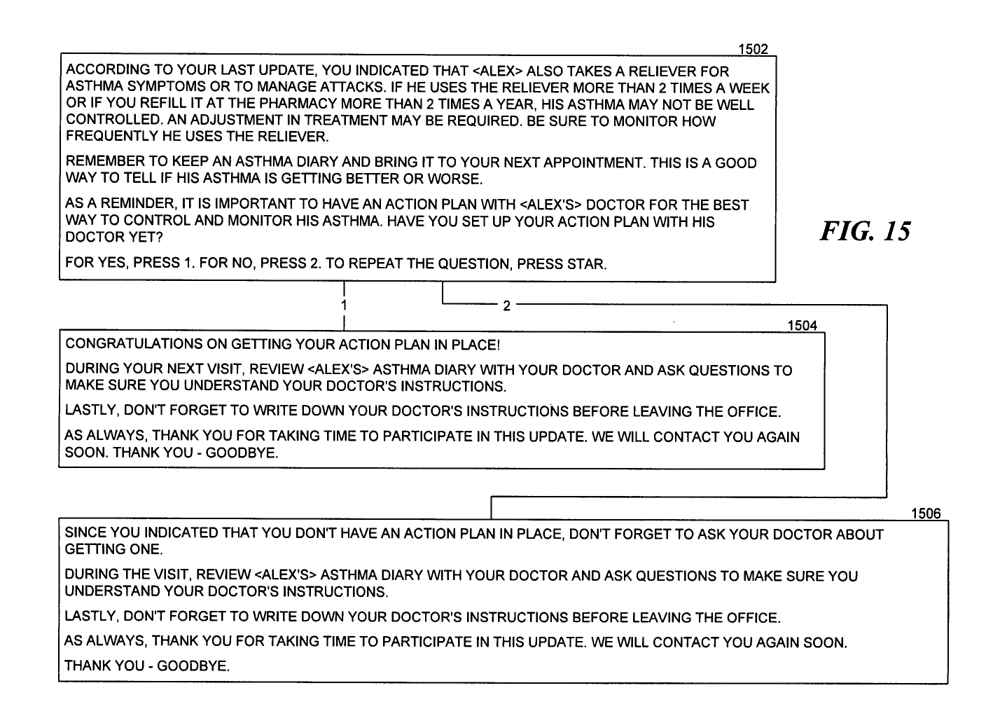

In FIG. 15 the healthcare management systems queries the patient about additional information related to the prescription and recommends follow-up medical services (process portion 1502). Depending on the response option selected by the patient, the healthcare management system provides tailored information regarding further instructions (process portions 1504 and 1506). Accordingly, the patient receives a reminder regarding follow-up medical care related to the prescription. In other embodiments, the healthcare management system can provide information related to newly prescribed medications (e.g., prescription counseling) and/or tips for remembering to take medication (e.g., a suggestion to keep the medication by the patient's toothbrush to aid the patient in remembering to take the medication). In still other embodiments, the healthcare management system can provide information and/or elicit responses to aid in patient monitoring. For example, in certain embodiments the healthcare management system can periodically contact a patient and provide a series of phone key selectable response options to determine the patient's mood (e.g., when the patient is taking a medication related to depression). In other embodiments, the healthcare management system can provide response options where the patient input will provide an indication of the presence or absence of drug related side effects.

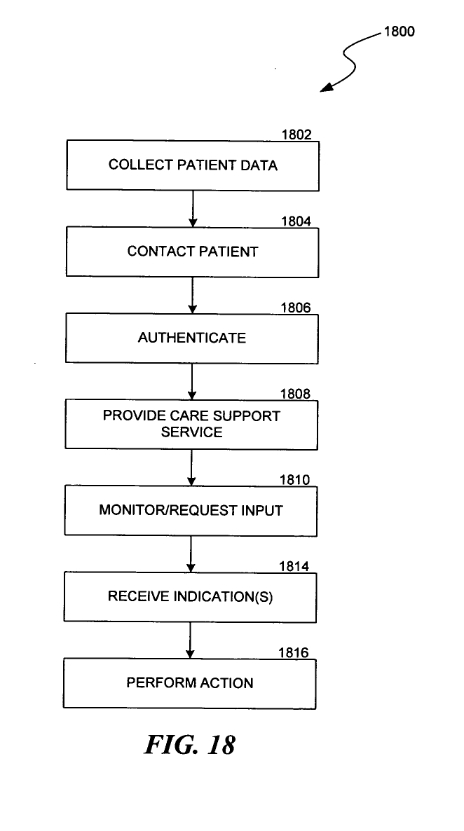

FIG. 18 illustrates a healthcare management process 1800, similar to that shown in FIG. 12, in accordance with other embodiments of the invention. The healthcare management process 1800 can include providing care support services related to one or more healthcare issues, such as providing treatment regimen adherence information and/or assistance in adhering to a healthcare regimen. For example, in certain embodiments the process 1800 in FIG. 18 can include receiving or collecting patent data (e.g., including any data associated with the patient, such as patient healthcare related data) for each one of multiple patients (process portion 1802) and contacting at least one of the patients (process portion 1804). The patient data can include a patient identification and/or information regarding one or more patient related healthcare issues.

For example, in selected embodiments the patient data can include information regarding an illness, disease, injury, long term wellness plan, selected medical condition, a prescribed treatment regimen, and/or the like. In selected embodiments, a prescribed or suggested treatment regimen can include a course of medication (e.g., including a prescription medication, an over the counter medication, a vitamin, and/or a supplement), an exercise regimen, a dietary regimen, a testing regimen, physical therapy or rehabilitation, any other medically related treatment regimen, and/or the like. In certain embodiments, a computing system can be used to collect or receive the patient data. For example, in selected embodiments the patient, a healthcare provider, a pharmacy, a caregiver, a third party, and/or the like can enter patient data into a computing system similar to the computing system discussed above with reference to FIG. 1, and the computing system can receive or collect the data.

The process 1800 can further include authenticating an identity of at least one patient (process portion 1806). In certain embodiments, the process of collecting patent data, the process of contacting at least one patient, and the process of the authenticating an identity of the at least one patient can be similar to the corresponding processes discussed above with reference to FIGS. 3-8.

In the illustrated embodiment, the process 1800 further includes providing care support service(s) to the at least one patient (process portion 1808), either directly or via a caregiver (e.g., a parent, family member, or other type of caregiver). In certain embodiments, the care support service can include information (e.g., care support information) related to the patient's one or more healthcare issues. For example, the care support service information can include, among other things, new medication counseling/information; a reminder to make a follow-up medical appointment related to a medication or treatment regimen; a reminder to take a medication and/or to adhere to a portion of a treatment regimen; a reminder regarding a scheduled consultation appointment; a tip on how to remember to take a medication; health education related to a medication, a treatment regimen, and/or a healthcare issue; testimonial information about a personal experience related to a treatment regimen and/or a healthcare issue; information related to drug side effects; information related to treatment regimen adherence (e.g., including drug dosage adherence monitoring); and/or the like. In other embodiments, care support service information can include providing information about related or relevant programs/groups based on the patient's one or more healthcare issues (e.g., smoking cessation programs, weight loss programs, nutrition education, Cancer Survivors Network, support groups, health education classes, and/or the like). In certain embodiments, the care support service information can include various response options that allow patient interaction so that the care support service information can be better tailored to the patient's needs.

In selected embodiments the process 1800 can further include monitoring and/or requesting an input (process portion 1810). For example, in selected embodiments a portion of a computing system can be used to monitor a patient's action(s) and/or a parameter related to the patient. In other embodiments, a computing system can request an input from a patient. For example, in certain embodiments a computing system can provided a patient with one or more input selections (e.g., via phone where the patient can respond to the input selections/queries using phone key selection(s), in a manner similar to that discussed above with reference to FIGS. 13-15). In still other embodiments, input can be requested from the patient via other methods (e.g., via some type of a text message).

In selected embodiments, the process 1800 can still further include receiving indication(s) (process portion 1814). For example, receiving an indication can include receiving input(s) from a patient (e.g., via computing system and/or in the form of phone key selection(s) corresponding to one or more input selection options provided to the patient by phone). In other embodiments, receiving indication(s) can include receiving input from an input device used to monitor a patient action (e.g., taking a medication, performing an exercise, performing a test procedure, using an aid device, and the like) and/or a healthcare related parameter (e.g., blood glucose level, blood pressure, blood oxygen saturation, and the like). In selected embodiments, receiving indication(s) can include not receiving an input when an input is expected. For example, if a computing system is performing a monitoring function and/or provides a patient with a query, the lack of an input or response can provide an indication that a certain event has not occurred. For example, receiving indication(s) can include not receiving an input that a patient has performed an expected or scheduled action, not receiving an input in response to a query that has been presented to the patient (e.g., via phone), and/or the like.

In still other embodiments, the process 1800 can include performing an action based on one or more received indication(s) (process portion 1816). For example, in certain embodiments information related to one or more of the indication(s) received from the patient can be provided to the patient or a third party, including a medical staff member (e.g., a pharmacy staff member and/or staff member at another type of medical related facility) or other individual/organization related to the well being of the patient (e.g., a patient's family member). In other embodiments, information related to the indication(s) received from the at least one patient can be summarized/analyzed and provided to the patient and/or a third party in the form of feedback (e.g., a “report card,” a reminder, etc.).

In selected embodiments, the healthcare management process 1800 can be used to provide care support services related to treatment regimen adherence. For example, collecting/receiving patient information (process portion 1802) can include collecting information regarding a prescribed or suggest treatment regimen and at least one of the patients (e.g., the patient and/or a patient's caregiver such as a parent, family member, or other type of caregiver) can be contacted (process portion 1804), for example, via phone. Providing care support service (process portion 1808) can include providing the patient with healthcare service information regarding the treatment regimen.

For instance, if medication and physical therapy exercises have been prescribed to treat a healthcare issue, the patient can be provided with information regarding the medication (e.g., new drug education), the physical therapy exercises, a related medical condition or healthcare issue, the importance of adhering to a medication dosing schedule, possible side-affects related to the medication, the importance of adhering to a physical therapy exercise schedule, and the like. Additionally, the patient can be reminded that a follow-up appointment should be made (e.g., to perform additional tests, to assess the effectiveness of the treatment, to monitor liver functions, other medically related appointments that are associated with and/or part of the treatment regimen). In selected embodiments, this process can be entirely automated by a computing system (e.g., healthcare service information can be provided by a recorded voice, a computer generated voice, and/or a visual message such as a text message or email).

In some embodiments, monitoring/requesting input (process portion 1808) can include providing the patient with option(s) (e.g., via a phone key interface) for being connected to a pharmacy, doctors office, or other healthcare related entity to schedule a follow-up appointment and/or to ask questions about the treatment regimen. In other embodiments, monitoring/requesting input (process portion 1808) can include providing the patient with option(s) to enroll in, schedule, or join a related program/group (as discussed above). Receiving indication(s) (process portion 1814) can include receiving an input selection from the patient indicating a selection of one or more options (e.g., via the phone key interface). In other embodiments, the healthcare management process 1800 can provide input options/requests and receive indication(s) in order to perform an automated scheduling or enrollment process (e.g., a computer based self-scheduling/enrollment system for scheduling follow-up appointments, enrolling in a support group, registering for a health education course, and/or the like).

In still other embodiments, various features discussed above can be provided to the patient to aid in treatment adherence. For example, the patient can be offered the opportunity to enroll in an automatic refill program, can be provided with refill reminders, can be provided with reminders that the patient has received the last refill available on a selected prescription, and/or provided with aid in obtaining a new prescription when an old prescription expires or has no more available refills (e.g., an option to be connected to a doctor's office and/or to have a pharmacy or other entity to contact the doctor's office on the patients behalf). In some cases, the information and convenience provided by the healthcare management system can positively influence the patient's adherence to a treatment regimen.

In certain embodiments, the healthcare management process 1800 can provide patient(s) with a survey (e.g., a series of questions that can be answered via a phone key interface or via another type of network connection) related to the likelihood that the patient will adhere to the treatment regimen. For example, providing care support service (process portion 1808) can include providing the patient with information regarding the treatment regimen and/or introductory information for the survey. Monitoring/requesting input (process portion 1810) can include providing survey questions related to understanding the health issue and treatment regimen, related to understanding the importance of adhering to the treatment regimen, related to understanding how to adhere to the treatment regimen, related to concerns about the treatment regimen (e.g., concerns about the cost of a medication and/or the potential side affects of a medication), related to the patient's success at adhering to past treatment regimen(s), related to the patient's memory abilities, and the like. Receiving input(s)/indication(s) (process portion 1814) can include receiving selected inputs (e.g., via a phone key interface) that answers the survey questions. Performing action(s) (process portion 1816) can include providing information related to the results of this survey to the patient and/or a third party (e.g., a medical service provider, medically related entity, a caregiver, a family member, or the like).

For example, in selected embodiments performing action(s) (process portion 1816) can include summarizing and/or analyzing the indication(s) from the survey to determine the patient's likelihood of adhering to the treatment regimen (e.g., the patients likely adherence can be rated on a scale of 1 to 10). In still other embodiments, performing an action (process portion 1816) can include summarizing/analyzing the results from the survey to determine what options and/or reminders should be suggested for, or provided to, the patient by the healthcare management process. For example, if the survey indicates that medication cost might be a factor in treatment regimen adherence, the healthcare management system might provide information to the patient regarding generic substitutions (e.g., as discussed above, in the form of a healthcare support service) and/or financial assistance (e.g., an assistance program provided by a pharmaceutical company and/or a government agency). In other embodiments, the healthcare management process might provide information about and/or options for obtaining a generic substitution (as discussed above).

In selected embodiments, the entire process can be implemented on a computing system. For example, based on the survey results various automated information and/or options can be provided to the patient and/or third party via recorded voice messages, computer generated voice messages, phone key interfaces, visual messages, email, text messages, and/or the like. Additionally, one skilled in the art will recognize that the health management system 1800 can include some or all of the process portions shown in FIG. 18 and/or multiples of the same process portion (e.g., performing various iterative processes).

In still other embodiments, the health management process 1800 can be used to provide reminders to perform portions of a treatment regimen (e.g., to take one or more medications, to perform and exercise therapy, to perform a monitoring test, and the like). For example, the patient can be contacted (process portion 1804) at a time that is appropriate for performing a selected portion of a treatment regimen (or periodically), the identity of the patient can be authenticated (process portion 1806), and care support service(s) (process portion 1808) can be provided. For example, the care support service can include a reminder to perform a task related to a treatment regimen.

In certain embodiments, the reminder can include a voice reminder provided via a phone network, a voice reminder provided by a dedicated device coupled to a network (e.g., a device 109 or wireless device 120 shown in FIG. 1), a visual reminder provided by a device coupled to a network, or the like. In selected embodiments, the healthcare management process 1800 can request input (process portion 1810) including requesting that the patient acknowledges the reminder (e.g., via a phone key interface/selection) and can receive indication(s) (process portion 1814) of this acknowledgement or lack thereof. In still other embodiments, the healthcare management process 1800 can provide a reminder regarding a scheduled appointment using a similar process.

In selected embodiments, the healthcare management process 1800 can provide the patient or a third party with the option to set up a reminder notification process for the patient. For example, in selected embodiments if adherence to a treatment program is expected to be a problem (e.g., based on a patient survey) the healthcare management process 1800 can provide care support service(s) (process portion 1808) in the form of information regarding a reminder notification process. The healthcare management process 1800 can then provide the patient (or a third party) with a request for input (process portion 1810) asking the patient (or third party) if the patient would like to receive/should receive notification reminders. The healthcare management process 1800 can then receive indication(s) (process portion 1814) related to this request for input and perform an action (process portion 1816) that includes scheduling or not scheduling reminder notifications based on the indication(s).

In selected embodiments the healthcare management process can provide the patient and/or a third party the opportunity to provide preferred contact information. For example, the healthcare management process 1800 can provide the patient (or a third party) with a request for input (process portion 1810) regarding preferred contact methods (e.g., phone numbers, email, etc.) and appropriate/preferred contact times. The healthcare management process 1800 can then receive indication(s) (process portion 1814) related to this request. For example, a patient might indicate that reminders and/or other information should be provided during the mornings or on weekends. Additionally, the patient might request that contact be established via a first phone number, and in the event that contact is not made, via a second phone number or via email. Similarly, third party contact information/preferences might be used for emergencies, follow-ups, and/or reporting, as discussed below in further detail. Of course, in some embodiments the appropriate reminder time or contact time may be determined or dictated by the treatment regimen and/or healthcare issue.

In yet other embodiments, the healthcare management process 1800 can include managing information regarding adherence to a healthcare treatment regimen. For example, in selected embodiments the healthcare management process can collect adherence information (e.g., monitor patient adherence), provide information that aids a patient in complying with a treatment regimen, provide information to the patient or a third party to aid in providing corrective action when there are adherence problems, and/or provide information based on the results of adherence to the treatment regimen. As discussed above, a healthcare treatment regimen can include a medication treatment regimen and/or other courses of treatment such as non-medication treatment regimens including rehabilitation exercises, other types of physical therapy/treatment, testing/monitoring activities, and the like.

For example, in certain embodiments patient data can be collected (process portion 1802) and can include patient identification information and/or information regarding a healthcare related issue and/or a healthcare treatment regimen (e.g., a treatment schedule including an exercise schedule, a testing schedule, a type of medication, a medication dosing schedule, and the like). In further embodiments, monitoring/requesting input (process portion 1810) can include monitoring the patient (e.g., via a computing system/network) to determine the patient's adherence to the healthcare treatment regimen. In still further embodiments, receiving indication(s) (process portion 1814) can include receiving an indication of adherence or none adherence to the treatment regimen.

In yet further embodiments, the healthcare management process can include performing an action (process portion 1816), for example, based on the patient's adherence or non-adherence to the treatment regimen. For instance, in selected embodiments an action can include providing information to the patient or a third party about the level of adherence obtained by the patient, ways to improve adherence, other types/recommended types of corrective action that might be required based on the level of adherence, a summary of test results (e.g., wherein the treatment regimen includes testing), recommended action based on test results, and the like. In certain embodiments, the action can include a cascading set of actions (e.g., elevating the level of action taken depending on the level of adherence to a treatment regimen and the action taken by the patient in response to previous recommendations provided by the healthcare management process 1800).

In selected embodiments where the treatment regimen includes a medication treatment regimen, receiving indication(s) can include receiving an indication that the patient has taken his or her medication and/or an indication that the patient has not taken his or her medication per the dosing schedule. For example, in selected embodiments the patient's medication can be carried in a smart pill box that can be monitored by a computing system (e.g., a device similar to device 109 or 120 shown in FIG. 1). When the pill box is opened, the computing system can receive an indication that the pill box was opened and/or that the medication was taken (e.g., if the smart pill box includes a feature where the patient indicates why the pill box was opened). The indication received by the computer can be compared to a medication dosing schedule to determine adherence to, or variations from, the medication dosing schedule. If the pill box does not provide an input that the medication was taken, the computing system thereby receives an indication that the medication was not taken according to the dosing schedule. In other embodiments, other devices can be used to provide indication(s) to a computing system. For example, the device can include any device configured to communicate with a computing system, including a device that allows the patient to indicate that the medication has been taken and/or allows the patient to provide some other type of input to the computing system (e.g., a text message, a voice input to a voice recognition module, and the like).

As discussed above, in certain embodiments the computing system can perform specific action based on the level of adherence indicated. For example, in selected embodiments the computing system can provide the patient an automated reminder or a report when a selected number (e.g., one or more) of dosages have been missed over a selected period of time. In some embodiments, the patient can be provided with an automated survey directed toward determining why medication dosages have been missed.

For example, the survey can include a phone key interface survey where the patient responds to automated survey response options via phone key selections. Based on the survey results, the computing system can provide tips on remembering to take his/her medication, offer the patient an option to receive future automated reminders, and/or send a report to a third party (e.g., care giver, family member, healthcare service provider, pharmacy, and/or the like). In still other embodiments, the computing system can request that the patient respond to the automated reminder or report, and if the computing system does not receive a response from the patient (e.g., receives an indication that the patient did not respond to the request) the computing system can provide an automated message to a third party indicating that the patient was unresponsive to the reminder, report, and/or survey.

In selected embodiments, the computing system can continue to monitor the smart pill box after the reminder, report, and/or survey have been provided to the patient. For example, if the patient continues to miss taking his or her medication per the dosing schedule, the computing system can provide additional reminders/reports, additional surveys, an automated message to a third party, and/or additional instructions (e.g., “go to the hospital now,” “call 911 and tell them that you need an ambulance,” etc.) In selected embodiments, the computing system can automatically contact 911 and provide an automated message requesting emergency assistance for the patient (e.g., when the level of medication treatment adherence is/could be life threatening). Accordingly, in certain embodiments the computing system can include an automated cascading and/or escalating series of actions/responses depending on the type of medication, the level of adherence to the dosing schedule, the responsiveness of the patient to previous reminders/aids, and/or the like. Although in the above example where the treatment regimen includes a medication treatment regimen, many or all of the process portions have been automated in a computer implemented process, in other embodiments fewer of the process portions are automated and/or performed by a computer implemented process.

In still other embodiments, a healthcare management process, similar to that discussed above, can be used to manage information regarding a non-medication related healthcare treatment regimen. For example, in selected embodiments receiving indication(s) can include receiving an indication that the patient has accomplished a specified task related to the healthcare treatment regimen. As discussed above, a specific task can include accomplishing one or more exercises (e.g., walking on a treadmill), performing a selected test (e.g., taking blood pressure, testing blood glucose levels, measuring a patient's weight, and/or the like). The indication(s) received can be compared to a treatment schedule or to other information regarding the treatment regimen (e.g., ranges associated with test results, performance parameters associated with an exercise, and/or the like). If an expected input is not received, the indication may be that the task was not performed.

Also as discussed above, in certain embodiments a specific action can be performed based on the adherence information (e.g., the received indication(s)). For example, in selected embodiments where the selected task includes a task having selected ranges or performance parameters (e.g., testing or exercising), the patient and/or a third party can be provided with the test results from a selected period of time (e.g., a summary and/or analysis of the test results). In still other embodiments, the patient and/or a third party can be provided with notification or a report when the results/performance of a selected task has a selected relationship to a certain range or performance expectation (e.g., when the results/performance parameters are abnormal). In some embodiments, based on the results/performance parameters of a selected task, the process can include providing additional instructions (e.g., go to the hospital now, call 911 and tell them that you need an ambulance, etc.). In other embodiments, the process can include contacting 911 and requesting emergency assistance for the patient (e.g., when the results/performance parameter is/could be life threatening).

In still other embodiments, a patient and/or a third party can be notified when a selected number of tasks have been missed over a selected period of time. In some embodiments, the patient can be provided with a survey directed toward determining why the tasks have been missed. For example, the survey can include a phone key interface survey where the patient responds to automated survey response options via phone key selections. Based on the survey results, the process can provide tips on remembering to perform the selected tasks, offer the patient an option to receive future reminders, and/or send a report to a third party. In selected embodiments, the process can continue to manage adherence information after an initial action has been taken (e.g., an initial reminder/report has been provided to a patient or third party), and a cascading and/or escalating series of responses or actions can be taken based on additional monitoring.

In various embodiments, any or all of the process portions in managing information regarding adherence to a healthcare treatment regimen can be computer implemented (e.g., the healthcare management process can be a computer implemented process) and/or completely automated. In other embodiments, various process portions can be manually performed. Additionally, in certain embodiments various device(s) configured to communicate with a computing system (similar to devices 109 and/or 120 in FIG. 1) can be used to monitor task performance and/or to provide indication(s) to a computing system.

For example, as discussed above, the device can include a smart pill box configured to communicate with a computing system (e.g., via a network) or any other device that allows the patient to indicate that the medication has been taken. In other embodiments an input device can include a selectable switch that allows a patient/user to send an input indicating a specified task has been performed (e.g., a medication has been taken, a test has been performed, and/or the like). In other embodiments, the device can include a device or computer that allows the patient to provide other type(s) of input to a computing system (e.g., a device configured to send a visual or text type message, a voice recognition module, and the like). In still other embodiments, the device can include a medical device configured to communicate with a computer. For example, in selected embodiments the device can include a scale, a blood glucose testing device, a blood pressure monitoring device, a treadmill, a Continuous Positive Airway Pressure (CPAP) machine, and/or the like.

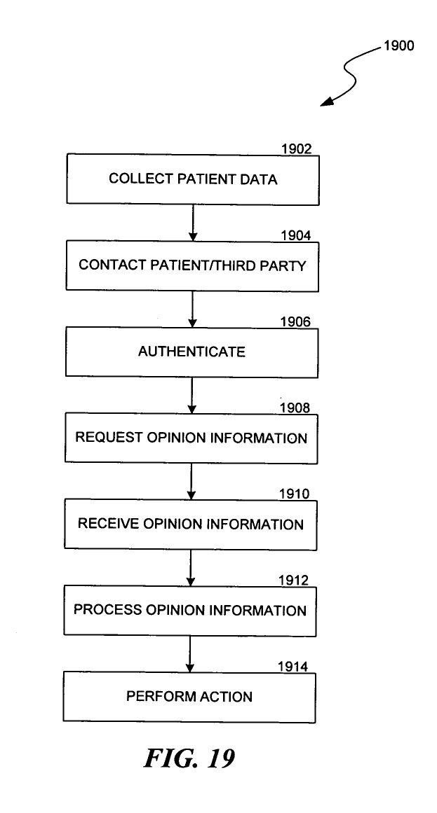

Additionally, as shown in FIG. 19, in certain embodiments a healthcare management process 1900 can be related to collecting opinion information. For example, in certain embodiments the healthcare management process 1900 can be used to collect opinion information regarding another healthcare management process (e.g., a healthcare management process similar to the healthcare management processes discussed above with reference to FIGS. 12-15 and 18). In other embodiments, the healthcare management process 1900 can be integral with another healthcare management process (e.g., process portions discussed below with reference to process 1900 can be include as part of other healthcare management processes discussed herein).

In certain embodiments, the healthcare management process 1900 can include collecting patient data (process portion 1902) in a manner similar to that discussed above with reference to FIG. 18. In selected embodiments, the patient data can include information regarding a related third party. For example, in certain embodiments the patient data can include information (e.g., contact information) related to a family member, caregiver, healthcare provider, guardian, or the like.

In selected embodiments, the patient and/or a third party can be contacted (process portion 1904). In certain embodiments, contact with the patient and/or a third party can be authenticated in a manner similar to that discussed above with reference to FIGS. 3-8. In selected embodiments, information can then be requested from the patient and/or third party in a manner similar to that discussed above with reference to FIGS. 9-11. For example, in certain embodiments information can be requested that is related to one or more of the healthcare management processes discussed above with reference to FIGS. 12-15, and 18.

For instance, in selected embodiments the patient and/or third party can be asked how useful treatment regimen reminders supplied by a management process have been in patient treatment regimen adherence and/or for suggestions on how to improve a healthcare management process. In other embodiments the patient and/or third party can be queried about overall customer satisfaction, satisfaction with specific aspects of a healthcare management process, satisfaction with a pharmacist or healthcare service provider, satisfaction with a consultation, and/or the like. In still other embodiments, opinion information can be collected that, after analysis, can provide an indication of the level of effectiveness a selected healthcare management process has on treatment regimen adherence.

In some embodiments, the opinion information can be requested in conjunction with another process portion. For example, in selected embodiments opinion information regarding treatment regimen reminders can be requested immediately after a reminder has been provided (e.g., via phone). In other embodiments, separate contact can be made with the patient/third party to request opinion information and/or a portal can be provided for receiving opinion information (e.g., a separate phone call providing an automated survey). For example, in selected embodiments a website or call-in number can be provided and patients/third parties desiring to provide patient opinion or feedback can provide the information via the portal.

In further embodiments, the healthcare management process can include receiving opinion information (process portion 1910), for example, the requested opinion information. In still further embodiments the process 1900 can include processing opinion information (process portion 1912), for example, summarizing opinion information, drawing conclusions from the opinion information, combining related opinion information from multiple patients/third parties, and/or the like. In yet other embodiments, the process 1900 can include performing an action (process portion 1914), for example, based on the opinion information that has been received.

For instance, in selected embodiments the opinion information (e.g., the raw information and/or the processed information) can be used to improve the process 1900 and/or alter the way the healthcare management process interacts with the patient/third party (e.g., change contact times for providing reminders, etc.). In other embodiments, the process information can be reported to various entities including related healthcare service providers (e.g., pharmacy, physician's office, support group, healthcare program providers/educators, insurance company, or the like. In still other embodiments, performing an action (process portion 1914) can include storing the opinion information, for example, for later use. In selected embodiments, the opinion information can include raw and/or processed opinion information and storing the opinion information can include storing the opinion information in a computing system (e.g., a database). In other embodiments, opinion information can be stored using other methods and/or techniques (e.g., opinion information can be stored in written and/or printed form).

Although in the illustrated embodiments many of the process portions are shown linked together, in other embodiments the process portions discussed above in FIGS. 12-15, 18, and 19 can stand alone, be accomplished as an iterative process, and/or be combined with other processes described herein. Additionally, as discussed above, the healthcare management system can include a computing system and some, all, or none of the care support service processes discussed above with reference to FIGS. 12-15, 18, and 19 can be performed in a computing environment. Additionally, although some of the embodiments of the care support service process discussed above with reference to FIGS. 12-15, 18, and 19 have been described using a phone key selection interface to receive input from the patient, those skilled in the art will understand that in other embodiments other methods of interfacing with the client can be used and/or that similar phone key selection interfaces can be used with other process portions described herein. For example, in other embodiments the healthcare management system can use voice recognition, email, fax, websites, and/or instant messaging to interface with patients.

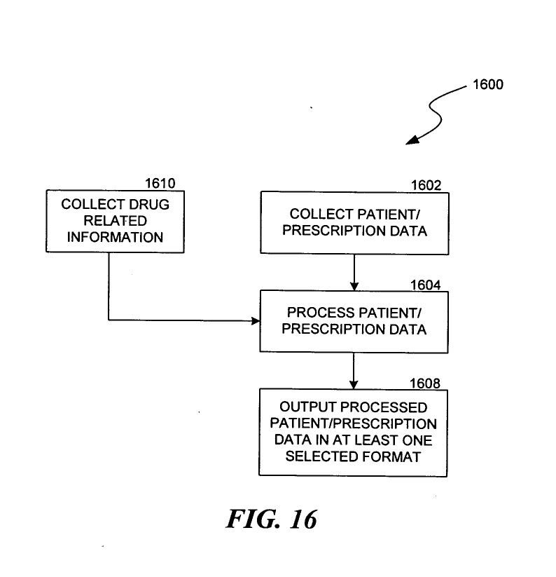

FIG. 16 is a flow diagram that illustrates a process for providing formatted prescription related data for use in a healthcare management element 1600 in accordance with still other embodiments of the invention. For example, in certain embodiments a healthcare management system can include a healthcare management element and an interface element. The interface element can collect raw or unformatted prescription related information (e.g., from an existing pharmacy database), process the information, and provide the formatted prescription related information to the healthcare management element. The healthcare management element can then contact one or more patients and can provide prescription related information to the patients (e.g., as discussed above with reference to FIGS. 2-15). For example, the healthcare management element can provide prescription fulfillment information, provides care support service information, and/or request patient opinion information.

In the illustrated embodiment, the process 1600 includes collecting raw or unformatted prescription related data for each one of multiple patients (process portion 1602). Each patient can be associated with one or more prescriptions and prescription related data can include at least one of the patient's identification and one or more drug names associated with the one or more prescriptions. Portions of the raw prescription related data can come from one or more sources and can have various formats. For example, the raw prescription related data can be collected from multiple pharmacies, multiple medical service providers/facilities, multiple databases, and/or multiple prescription related systems. In selected embodiments, at least a portion of the raw prescription related data can come from various existing (e.g., legacy) pharmacy systems or databases, such as TechRX™, available from the NDCHealth® Corporation of Atlanta, Ga.; PDX, available from PDX, Inc. of Fort Worth, Tex.; Synercom™, available from HCC of Fort Worth, Tex.; and ScriptPro®, available from ScriptPro LLC of Mission, Kans.

Because the raw prescription related data may not be in a format that is usable by a healthcare management element, it can be necessary to process the raw prescription related data in order to place the data in a format that the healthcare management element can use. Accordingly, the process 1600 can include processing the raw prescription related data (process portion 1604) to provide formatted prescription related data that is usable by a healthcare management element. In certain embodiments, processing the prescription related data can include combining various portions of prescription related data, aggregating a portion of prescription related data, parsing a portion of the prescription related data, summarizing a portion of prescription related data, coding the prescription related data to be readable/usable by a healthcare management element, and/or filtering a portion of prescription related data. Filtering prescription related data can include various functions, such as removing duplicate data entries and/or scrubbing the data to remove extraneous information.

In other embodiments, the process 1600 can include collecting drug-related information (process portion 1610) and the drug-related information can be used to supplement and/or filter the raw prescription related data. For example, in certain embodiments, during prescription related data processing the prescription related data can be filtered against the drug related information. For instance, the prescription related data can be filtered against the drug-related information to determine an availability of a drug associated with one or more of the prescriptions, a cost of the drug associated with one or more the prescriptions, an availability of a generic drug related to a medication associated with one or more of the prescriptions, a cost of a generic drug related to a medication associated with one or more of the prescriptions, whether a drug associated with one or more of the prescriptions is being recalled, a side effect associated with the drug associated with one or more the prescriptions, and/or a potential for interaction between a first prescription associated with a selected patient in a second prescription associated with the selected patient.

The formatted prescription related data can then be provided for use by a healthcare management element (process portion 1608). For instance, the process 1600 can be used to provide formatted prescription related data to a healthcare management element that provides prescription fulfillment information, provides care support service information, and/or collects patient opinion information. Additionally, as discussed above, the healthcare management system can include a computing system and some, all, or none of the processes for providing formatted prescription related data for use in a healthcare management element (discussed above with reference to FIG. 16) can be performed in a computing environment.

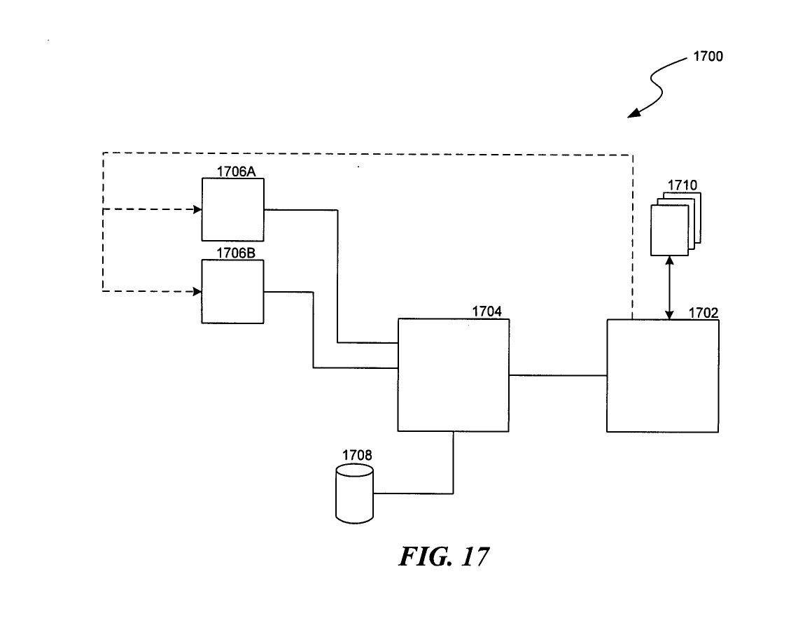

FIG. 17 is a partially schematic illustration of a healthcare management system 1700 suitable for implementing various embodiments of the invention. In FIG. 17, the healthcare management system 1700 includes a healthcare management element 1702 and an interface element 1704. The healthcare management element 1702 can be configured to communicate with one or more patients 1710 (e.g., via a patient's phone system and/or computing system). Each patient can be associated with one or more prescriptions, and the healthcare management element 1702 can provide information to, and/or collect information from, the patient's 1710. For example, in selected embodiments the healthcare management element can be configured to provide prescription fulfillment information, provide care support service information, and/or provide information requesting patient opinion information. As discussed above, in various embodiments the raw prescription related data and/or the formatted prescription related data can include at least one of the patient's identification associated with one or more prescriptions and one or more drug names associated with the one or more prescriptions.

The interface element 1704 can be operatively coupled to the healthcare management element 1702 to provide formatted prescription related data to the healthcare management element 1702. The interface element 1704 can include a computing element or system that is configured to collect raw or unformatted prescription related data that would be unusable by the healthcare management element, process the raw prescription related data, and provide formatted prescription related data to the healthcare management element 1702 (e.g., formatted prescription related data that is formatted to be usable by the healthcare management element 1702). For example, in various embodiments the interface elements 1704 can process the raw prescription related data as discussed above with reference to FIG. 16.

As discussed above, the raw prescription related data can be gathered from various and/or multiple sources (e.g., from existing or legacy databases of various medical service providers). For example, in FIG. 17 two pharmacies (shown as a first pharmacy 1706a and a second pharmacy 1706b) are shown coupled to the interface element 1704 to provide raw prescription related data to the healthcare management system 1700. Additionally, in the illustrated embodiment the healthcare management element 1702 of the healthcare management system 1700 is configured to provide output (e.g., information or data) to the pharmacies 1706a and 1706b (e.g., based on patient selections).

For example, as discussed above, in certain embodiments the healthcare management system 1700 can provide raw data collected by the system and/or analytics of the collected data to a medical service provider. This information or data can be used for various purposes including providing information that will allow the medical service provider to better serve patient needs and/or identify areas where the medical service provider needs to improve performance. Additionally, providing data analytics can include providing formatted or summarized information. For example, in certain embodiments the data analytics can provide information to a medical service provider that is summarized according to various demographics (e.g., age, race, sex, drug, insurance coverage, and/or medical condition) and/or other common characteristics.

In certain embodiments, the interface element 1704 can also collect drug-related information from various databases. For example, in certain embodiments the interface element 1704 can collect data from a drug information service such as Medi-Span, available form Wolters Kluwer Health, Inc. of Indianapolis, Ind. As discussed above with reference to FIG. 16, the drug-related information can be used to supplement and/or filter the raw prescription related data.

A feature of embodiments discussed above is that a healthcare management system can provide an effective and efficient way to accomplish many of the day-to-day pharmacy tasks that are time-consuming and expensive. Accordingly, selected embodiments of a healthcare management system can reduce pharmacy operating costs, allow a pharmacy to provide a higher quality of service to its patients, and/or allow a pharmacy to service a larger number of patients. This feature not only can result in a cost savings and/or better health care services, but can provide pharmacy staff members with higher levels of job satisfaction because they can more effectively focus on patient needs.

From the foregoing, it will be appreciated that specific embodiments of the invention have been described herein for purposes of illustration, but that various modifications may be made without deviating from the invention. For example, although various embodiments have been discussed above in the context of a pharmacy or a pharmaceutical provider, one skilled in the art will understand that these embodiments are equally applicable to any healthcare related organization that interfaces with patients. Additionally, aspects of the invention described in the context of particular embodiments may be combined or eliminated in other embodiments. For example, although advantages associated with certain embodiments of the invention have been described in the context of those embodiments, other embodiments may also exhibit such advantages. Furthermore, not all embodiments need necessarily exhibit such advantages to fall within the scope of the invention. Accordingly, the invention is not limited except as by the appended claims.

I/We claim:

1. A method for providing care support services related to one or more healthcare issues, comprising:
collecting patient data for each one of multiple patients, the patient data including at least one of a patient identification and information regarding one or more healthcare issues;
contacting at least one of the patients;
providing one or more response options regarding the at least one patient's one or more healthcare issues;
receiving an input indicating a selection of at least one of the one or more response options; and
providing information regarding the selection of at least one of the one or more response options to (a) the at least one patient, (b) a third party, or (c) both (a) and (b).

2. The method of claim 1 wherein:
collecting patient data includes collecting medication related data for each one of multiple patients, each patient being associated with one or more medications, the medication related data including at least one of a patient identification and one or more drug names associated with the one or more medications;
providing one or more response options includes providing one or more response options to the at least one of the patient regarding medication regimen adherence; and
providing information regarding the selection of at least one of the one or more response options includes providing information regarding (a) medication regimen adherence, (b) the likelihood that the at least one patient will adhere to the medication regimen, or (c) both (a) and (b).

3. The method of claim 1 wherein a method for providing care support services related to one or more healthcare issues includes a method in a computing environment.

4. The method of claim 1 wherein a method for providing care support services related to one or more healthcare issues includes a method in a computing environment, and wherein:
contacting at least one of the patients includes contacting at least one of the patients via phone;
providing one or more response options includes providing one or more response options via phone; and
receiving an input includes receiving an input via phone.

5. The method of claim 1 wherein the at least one patient's one or more healthcare issue includes at least one of a patient's mood, a presence or non-presence of one or more selected side effects, an indication of the effectiveness or non-effectiveness of the medication, information regarding adherence to a treatment regimen, and a level of control of a chronic condition.

6. The method of claim 1 wherein information regarding adherence to a treatment regimen includes information regarding adherence to a medication adherence regimen.

7. The method of claim 1, further comprising:
receiving opinion information related to the information regarding the selection of at least one of the one or more response options; and
performing an action based on the opinion information.

8. A method for providing care support services related to one or more healthcare issues, comprising:
collecting patient data for each one of multiple patients, the patient data including at least one of a patient identification and information regarding one or more healthcare issues;
contacting at least one of the patients; and
providing a care support service to the at least one of the patients related to the at least one of the patient's one or more healthcare issues.

9. The method of claim 8 wherein a method for providing care support services related to one or more healthcare issues includes a method in a computing environment.

10. The method of claim 8 wherein a method for providing care support services related to one or more healthcare issues includes a method in a computing environment, and wherein:
contacting at least one of the patients includes contacting at least one of the patient via phone; and
providing care support service to the at least one of the patients includes providing care support service to the at least one of the patients via phone.

11. The method of claim 8 wherein providing the care support service includes providing at least one of new medication counseling, a medical follow up reminder, a reminder to take a prescription, a tip for remembering to take a prescription, health education, testimonial information about a personal experience, information related to dosage adherence monitoring, information regarding adhering to a healthcare treatment regimen, information related to drug side effects, and information related to patient monitoring.

12. The method of claim 8, further comprising:
providing one or more response options related to the care support service; and
receiving an input indicating a selection of at least one of the one or more response options.

13. The method of claim 8, further comprising:
receiving opinion information related to the care support service; and
performing an action based on the opinion information.

14. A method for managing information regarding adherence to a treatment regimen, comprising:
collecting patent data for each one of multiple patients, the patient data including at least one of a patient identification and information regarding one or more healthcare issues;
receiving an indication regarding at least one of the patient's adherence to a non-medication treatment regimen; and
providing information regarding the indication to (a) the at least one patient, (b) a third party, or (c) both (a) and (b).

15. The method of claim 14 wherein the method of collecting information regarding adherence to a treatment regimen includes a method in a computing environment.

16. The method of claim 14, further comprising monitoring a device associated with at least one of the patients for the indication.

17. The method of claim 14 wherein providing information regarding the indication includes providing information regarding a level of adherence to at least a portion of the treatment regimen.

18. The method of claim 14, further comprising:
receiving opinion information related to the information regarding the indication; and
performing an action based on the opinion information.

19. A method in a computing environment for managing information regarding adherence to a treatment regimen, comprising:
collecting patient data for each one of multiple patients, the patient data including at least one of a patient identification and information regarding one or more healthcare issues;
receiving an indication regarding at least one of the patient's adherence to a medication treatment regimen; and
providing information regarding the indication to (a) the at least one patient, (b) a third party, or (c) both (a) and (b).

20. The method of claim 19, further comprising monitoring a device associated with at least one of the patients for the indication.

21. The method of claim 19, wherein providing information includes providing an automated voice message, an automated text message, or both.

22. The method of claim 19 wherein providing information regarding the indication includes providing information regarding a level of adherence to at least a portion of the treatment regimen.

23. The method of claim 19 wherein receiving a selected input includes receiving a selected input from a smart pill box associated with a patient and providing information regarding the indication includes providing information regarding a level of adherence to at least a portion of a medication treatment regimen.

24. The method of claim 19, further comprising:
receiving opinion information related to the information regarding the indication; and
performing an action based on the opinion information.
